(19) United States
(12) Patent Application Publication
(10) Pub. No.: US 2025/0388928 A1
(43) Pub. Date: Dec. 25, 2025

(54) PRECISION RECOMBINANT ADENO-ASSOCIATED VIRUS VECTOR AND USE THEREOF

(71) Applicant: SUZHOU GENEHEALTH BIOTECHNOLOGY,CO., LTD., KUNSHAN CITY, JIANGSU PROVINCE (CN)

(72) Inventors: Jiahong SHAO, Kunshan (CN); Xiang WU, Kunshan (CN); Pengcheng TAN, Kunshan (CN); Xiaoming ZHAO, Kunshan (CN); Zhenzhen LEI, Kunshan (CN); Yang LU, Kunshan (CN); Tingjun XUN, Kunshan (CN)

(21) Appl. No.: 18/879,723

(22) PCT Filed: Jun. 30, 2023
(86) PCT No.: PCT/CN2023/104702

§ 371 Date: Dec. 27, 2024

(30) Foreign Application Priority Data
Jun. 30, 2022 (CN) 202210762934.3

(51) Int. Cl. C12N 15/86

(52) U.S. Cl. CPC C12N 15/86; C12N 2710/14021; C12N 2750/14143; C12N 2750/14152

(57) ABSTRACT

A precision recombinant adeno-associated virus (pciAAV) vector and the use thereof in gene therapy, gene editing, and gene regulation. The pciAAV vector is obtained by packaging an unpackaged pciAAV genome, said pciAAV genome containing the following, in sequence: (a) a modified ITR, which lacks a D element and a trs sequence; (b) a gene of interest or a protection sequence; (c) a complete ITR; (d) a gene of interest or a protection sequence; and (e) a modified ITR, which lacks a D element and a trs sequence; wherein at least one of the segments (b) and (d) contains a gene of interest. In the present invention, the level of impure DNA in the AAV vector is greatly reduced, gene expression efficiency is improved, random integration in the AAV gene vector is reduced, and the risk of gene mutation is reduced by the precision recombinant adeno-associated virus (pciAAV) vector.

### TECHNICAL FIELD

The present invention generally relates to recombinant adeno-associated virus (rAAV) vectors, in particular, to precision recombinant adeno-associated virus (pciAAV) vectors and use thereof in gene therapy, gene editing, and gene regulation.

### BACKGROUND

Gene therapy addresses patients with genetic mutations caused by abnormal gene expression profiles, including the treatment or prevention of genetic diseases resulting from gene defects, abnormal regulation, or expression, such as underexpression- or overexpression-induced disorders and malignancies. Treatment, prevention or remission of the diseases can be achieved by delivering corrective genetic material to the patient.

Currently, gene delivery vectors primarily include non-viral vectors and viral gene delivery vectors. Among various available virus-derived gene vectors (e.g., recombinant retrovirus, recombinant lentivirus, recombinant adenovirus, etc.), recombinant adeno-associated virus (rAAV) gene vectors are increasingly favored.

Adeno-associated virus (AAV) belongs to the family Parvoviridae. With advantages such as non-pathogenicity, low immunogenicity, and broad infectivity, AAV has become the most promising gene vector for gene therapy. AAV is a non-enveloped single-stranded DNA virus carrying an approximately 4.7 kb linear single-stranded DNA genome. The two ends of the AAV DNA genome feature 145 bp inverted terminal repeats (ITRs), of which the terminal 125 bases form a longer palindromic structure capable of self-folding via complementary base pairing, presenting a T-shaped hairpin structure. The positive strand and negative strand DNA genomes carrying wild-type ITRs are packaged into the AAV viral capsid with equal probability, thereby producing equal quantities of positive strand or negative strand DNA AAV viral particles. AAV viral particles infect cells, enter the nucleus, uncoat, and release the AAV genome into the nucleus. The ITRs at both ends of the AAV genome may fold into T-shaped hairpin structures, where the 3′ end serves as a primer for DNA synthesis to generate a second strand, forming a double-stranded DNA molecule that initiates the expression of genes carried by the AAV genome. The positive strand and strand DNA molecules of the AAV genome can also complement each other to form double-stranded DNA molecules, enabling gene expression. Furthermore, inter-molecular ITRs spontaneously combine to form dimers and multimers, which allow for the long-term or even lifelong expression of exogenous genes in cells.

The ITRs at both ends of the AAV DNA genome contain essential information for AAV DNA replication, packaging, integration, and rescue. Traditional rAAV packaging systems rely on the ITRs of AAV as the basic packaging elements. Specifically, two ITRs are positioned at both ends of the DNA to be packaged into the rAAV capsid, namely D-trs-A′-C′-C-B′-B-A at the 3′ end of the packaging gene DNA strand and A′-B′-B-C′-C-A-trs-D at the 5′ end. These are used to package the DNA into the rAAV capsid. Since its inception, this rAAV vector packaging system has been in use for decades and faces challenges such as outdated technology, impure rAAV vector DNA molecules (containing 3-6% plasmid backbone impurity DNA), low quality, low gene expression efficiency, and significant clinical toxic side effects. Moreover, this packaging design produces single-polar single-stranded rAAV viral particles lacking ITRs at the 3′ end and containing plasmid backbone impurity DNA, which may cause insertional mutations or severe medical incidents in clinical applications, severely impacting the utility of rAAV vectors.

In summary, conventional packaging of rAAV vectors suffers from issues such as low DNA molecule purity, low gene expression efficiency, poor gene operability, and risks of insertional mutations, significantly limiting their use.

### SUMMARY OF THE INVENTION

Provided herein is a precision DNA recombinant adeno-associated virus (pciAAV) vector. Through extensive research, the inventors have discovered that the pciAAV vector as described herein can reduce or eliminate impurity DNA (e.g., plasmid backbone impurity DNA) from being packaged into the AAV capsid, which facilitates improvements in rAAV gene packaging efficiency, rAAV gene expression efficiency, and the effectiveness and safety of rAAV-based gene therapy, thereby greatly promoting the development and use of rAAV vectors and rAAV-based gene therapy.

In one aspect, provided herein is an unpackaged pciAAV genome, which comprises the following, in sequence:
(a) a modified ITR that lacks a D element and a trs sequence;
(b) a gene of interest or a protection sequence;
(c) a complete ITR;
(d) a gene of interest or a protection sequence; and
(e) a modified ITR that lacks a D element and a trs sequence;
wherein at least one of the segments (b) and (d) comprises a gene of interest.

In another aspect, provided herein is a pciAAV transgenic plasmid comprising the unpackaged pciAAV genome as described herein.

In another aspect, provided herein is a pciAAV vector comprising:
a capsid protein; and a pciAAV genome packaged by the capsid protein;
wherein the pciAAV genome packaged by the capsid protein is derived from the unpackaged pciAAV genome as described herein.

In another aspect, provided herein is a method for packaging a pciAAV vector, comprising: transforming DH10Bac competent E. coli cells with the pciAAV transgenic plasmid as described herein and with Cap and Rep expression plasmids, respectively; performing at least one round of blue-white colony screening, picking white colonies, amplifying, and extracting recombinant bacmids; transfecting insect cells with the recombinant bacmids to produce recombinant baculovirus; and extracting the recombinant baculovirus and infecting insect cells with the recombinant baculovirus to obtain the pciAAV vector.

In another aspect, provided herein is a method for delivering a gene of interest (GOI) to cells, comprising: contacting the cells with one or more pciAAV vectors as described herein; wherein the genome of the one or more pciAAV vectors comprises a gene expression cassette for the GOI and/or optionally other DNA sequences; wherein the pciAAV vector is packaged from the unpackaged pciAAV genome as described herein.

In another aspect, provided herein is an isolated host cell comprising one or more pciAAV vectors as described herein.

In another aspect, provided herein is the use of the one or more pciAAV vectors in gene expression.

In another aspect, provided herein is the use of the one or more pciAAV vectors in gene therapy.

In another aspect, provided herein is the use of the one or more pciAAV vectors in gene editing.

In another aspect, provided herein is the use of the one or more pciAAV vectors in gene regulation.

### DESCRIPTION OF DRAWINGS

Provided below is a further explanation of the invention in conjunction with the figures, which are presented solely to illustrate embodiments of the invention and not to limit the scope of the invention.

FIG. 1 illustrates a schematic diagram of the DNA sequence structure of AAV2 Flip ITR (showing RBE and trs sites).

FIG. 2 illustrates a schematic diagram of the construction and packaging design of a pciAAV vector according to an exemplary embodiment described herein.

FIG. 3 shows the SDS-PAGE results of capsid proteins of a pciAAV vector according to an exemplary embodiment described herein. Lane MW: protein molecular weight standard; Lane 1: rAAV-EGFP vector; Lane 2: pciAAV-EGFP vector.

FIG. 4 shows the results of neutral agarose gel electrophoresis of a pciAAV vector according to an exemplary embodiment described herein. Lane MW: DNA molecular weight standard; Lane 1: rAAV-EGFP vector; Lane 2: pciAAV-EGFP vector; Lane 3: 4.7 kb PCR DNA fragment.

FIG. 5 shows the results of alkaline agarose gel electrophoresis of a pciAAV vector according to an exemplary embodiment described herein. Lane MW: DNA molecular weight standard; Lane 1: pciAAV-EGFP vector; Lane 2: rAAV-EGFP vector; Lane 3: 4.7 kb PCR DNA fragment.

FIG. 6 illustrates a schematic diagram of primer design for impurity DNA PCR analysis of a pciAAV vector according to an exemplary embodiment described herein.

FIG. 7 shows the results of PCR analysis of impurity DNA for a pciAAV vector according to an exemplary embodiment described herein. FIG. 7A: PCR products of the pciAAV vector. Lane MW: DNA molecular weight standard; Lane 1: pciAAV-EGFP plasmid, primers F1 and R1; Lane 2: pciAAV-EGFP plasmid, target DNA template primers; Lane 3: pciAAV-EGFP plasmid, primers F2 and R2; Lane 4: pciAAV-EGFP empty particle, primers F1 and R1; Lane 5: pciAAV-EGFP empty particle, target DNA template primers; Lane 6: pciAAV-EGFP empty particle, primers F2 and R2; Lane 7: pciAAV-EGFP vector, primers F1 and R1; Lane 8: pciAAV-EGFP vector, target DNA template primers; Lane 9: pciAAV-EGFP vector, primers F2 and R2. FIG. 7B: PCR products of the rAAV vector. Lane MW: DNA molecular weight standard; Lane 1: rAAV-EGFP plasmid, primers F1 and R1; Lane 2: rAAV-EGFP plasmid, target DNA template primers; Lane 3: rAAV-EGFP plasmid, primers F2 and R2; Lane 4: rAAV-EGFP empty particle, primers F1 and R1; Lane 5: rAAV-EGFP empty particle, target DNA template primers; Lane 6: rAAV-EGFP empty particle, primers F2 and R2; Lane 7: rAAV-EGFP vector, primers F1 and R1; Lane 8: rAAV-EGFP vector, target DNA template primers; Lane 9: rAAV-EGFP vector, primers F2 and R2.

FIG. 8 shows the results of gene expression analysis for HEK293 cells transfected with a pciAAV vector according to an exemplary embodiment provided herein. A: green fluorescence image of HEK293 cells transfected with a pciAAV vector. B: green fluorescence image of HEK293 cells transfected with an rAAV vector. C: flow cytometry analysis of transfection efficiency and gene expression efficiency in HEK293 cells.

### DETAILED EMBODIMENTS

The meanings of technical terms in the application are consistent with the general understanding of those skilled in the art, unless otherwise specified. In the application, “a” or its combinations with various quantifiers include both singular and plural meanings unless specifically stated otherwise. In the application, for the same parameter or variable, when multiple values, value ranges, or their combinations are provided for illustration, it is equivalent to specifical disclosure of those values, the range endpoints, and value ranges formed by any combination of them. In the application, any value, whether modified by terms such as “about” or not, covers an approximate range understood by those skilled in the art, such as ±10%, ±5%, etc. Each “embodiment” described herein equally refers to and encompasses embodiments of the methods and systems of the application. In the application, one or more technical features of any embodiment can be freely combined with one or more technical features of any other embodiment, and the resulting embodiments are also considered part of the present disclosure.

Adeno-associated virus (AAV) is a member of the Parvoviridae family. It is non-enveloped and exhibits icosahedral symmetry. To date, over ten serotypes and hundreds of variants of AAV have been identified. Among them, AAV2 is the most thoroughly studied and widely applied serotype.

The AAV genome comprises a linear single-stranded DNA of approximately 4.7 kb in length, flanked at both ends by inverted terminal repeats (ITRs) of approximately 145 bases long. Within the ITRs, about 125 bases near the ends contain palindromic sequences that can fold back on themselves through complementary base pairing to form T-shaped hairpin structures. The ITRs comprise Rep binding elements (RBEs) and terminal resolution sites (trs), which can be recognized and bound by Rep proteins and cleaved at the trs.

The AAV genome contains two open reading frames (ORFs) located between the ITRs at both ends. These two ORFs encode rep and cap. The rep gene encodes four Rep proteins: Rep78, Rep68, Rep52, and Rep40, which function in AAV replication, integration, rescue, and packaging. The cap gene encodes the AAV capsid proteins VP1, VP2, and VP3. Among them, VP1 is essential for forming infectious AAV, while VP3 is the primary protein for forming the AAV viral particles. The ratio of VP1, VP2, and VP3 in the AAV capsid is approximately 1:1:10.

During AAV assembly into viral particles, the positive strand and negative strand DNA genomes carrying wild-type ITRs are packaged into the AAV capsid with equal probability, resulting in equal amounts of positive strand and negative strand AAV viral particles. AAV viral particles infect cells, uncoat within the cells, and release the AAV genome. After the ITRs at both ends of the AAV genome fold into T-shaped palindromic hairpin structures, the 3′ end serves as a primer for synthesizing the second strand, forming a double-stranded molecule that subsequently initiates the expression of genes carried by the AAV genome.

The positive strand and negative strand DNA molecules of the AAV genome can also complement each other to form double-stranded DNA, enabling gene expression.

Unpackaged pciAAV Genome

In one aspect, provided herein is an unpackaged pciAAV genome, which comprises the following, in sequence:
(a) a modified ITR that lacks a D element and a trs sequence;
(b) a gene of interest or a protection sequence;
(c) a complete ITR;
(d) a gene of interest or a protection sequence; and
(e) a modified ITR that lacks a D element and a trs sequence;
wherein at least one of the segments (b) and (d) comprises a gene of interest.

As used herein, the term “recombinant adeno-associated virus” or “rAAV” vector refers to a non-wild-type adeno-associated virus particle comprising a recombinant AAV genome packaged within a viral capsid, serving as a gene delivery vector.

As used herein, the term “precision DNA recombinant adeno-associated virus” or “pciAAV” vector refers to an rAAV vector provided herein that is designed and optimized to enable precise DNA packaging and delivery. In some cases, “precise DNA packaging” refers to the rAAV vector (e.g., pciAAV vector) formed by packaging, which has reduced or eliminated levels of impurity DNA (e.g., plasmid backbone impurity DNA). This reduction or elimination of impurity DNA delivery enhances rAAV gene packaging efficiency, gene expression efficiency, and the effectiveness and safety of rAAV-based gene therapy.

As used herein, the term “unpackaged pciAAV genome” refers to a recombinant AAV genome intended for packaging (e.g., for cloning into a plasmid vector) and subsequently used for packaging into capsid proteins.

As used herein, the term “complete ITR” generally refers to the 145-base ITR sequence found in the traditional AAV2 genome, typically comprising D, A′, C′, C, B′, B, and A elements, as well as RBE and trs sites.

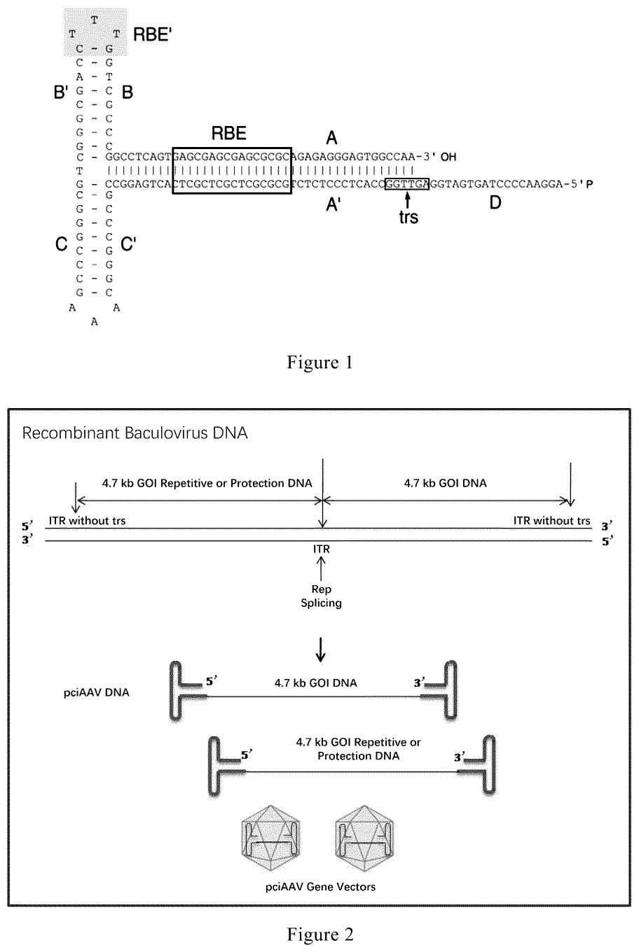

It should be understood that the complete ITR sequences at the ends of the AAV genome may have identical or different orientations, such as Flip/Flop, Flop/Flip, Flip/Flip, or Flop/Flop. An exemplary structural orientation of a “Flip” ITR is shown in FIG. 1, which generally includes the D element, A′ element, C′ element, C element, B′ element, B element, and A element, with trs and RBE sites. An exemplary structural orientation of a “Flop” ITR is shown in FIG. 1, except that the B′ segment and C′ segment swap positions, and the B segment and C segment swap positions.

As used herein, the term “modified ITR” generally refers to an ITR derived from a complete ITR through modification (e.g., truncation). In some embodiments, the modified ITR lacks the D element and trs sequence compared to the complete ITR.

As used herein, the term “hairpin structure”, also referred to as a “hairpin loop structure”, refers to a “hairpin”-like structure formed by a DNA molecule folding back on itself, with complementary base pairing occurring within the folded region. Examples include T-shaped hairpin structures, as shown in FIG. 1.

In some embodiments, the unpackaged pciAAV genome described herein comprises the above segments (a) to (e) in 5′ to 3′ direction.

As used herein, the terms “gene of interest (GOI),” “gene to be delivered,” “exogenous gene,” “exogenous nucleic acid,” or “target gene” are used interchangeably and refer to a gene originating from outside the organism of interest or research or a gene intended for delivery to the organism. The GOI may be any gene desired to express or produce a biological function in the recipient cells to which the pciAAV viral particles will be delivered.

In some embodiments, the GOI comprises a nucleotide sequence encoding the GOI. In some embodiments, the nucleotide sequence encoding the GOI is a DNA sequence.

In some embodiments, the GOI includes filler sequences at one end (e.g., the 5′ or 3′ end) or both ends (e.g., the 5′ and 3′ ends). In some embodiments, filler sequences are included between the modified ITR (e.g., segment (a) or (e)) and the complete ITR (e.g., segment (c)). “Filler sequence” generally refers to a nucleotide sequence contained within a larger nucleic acid molecule (e.g., a plasmid vector), typically used to create a desired spacing between two nucleic acid elements (e.g., between a promoter and a coding sequence such as the GOI) or to extend the nucleic acid molecule to a desired length. Filler sequences do not contain protein-coding information and may have an unknown or synthetic origin and/or be unrelated to other nucleic acid sequences within the larger nucleic acid molecule.

In some embodiments where the DNA sequences of some GOIs are shorter than the AAV packaging length of 4.7 kb, filler sequences are added as auxiliary DNA to extend the DNA to be packaged into a single viral particle to the AAV packaging length (e.g., approximately 4.7 kb).

In some embodiments, the spacing between the modified ITR (e.g., segment (a) or (e)) and the complete ITR (e.g., segment (c)) is approximately 4.7 kb (e.g., at least 4.0 kb, at least 4.1 kb, at least 4.2 kb, at least 4.3 kb, at least 4.4 kb, at least 4.5 kb, at least 4.6 kb, up to 4.7 kb). In some embodiments, filler sequences are included to achieve a spacing of approximately 4.7 kb (e.g., at least 4.0 kb, at least 4.1 kb, at least 4.2 kb, at least 4.3 kb, at least 4.4 kb, at least 4.5 kb, at least 4.6 kb, up to 4.7 kb) between the modified ITR (e.g., segment (a) or (e)) and the complete ITR (e.g., segment (c)).

In some embodiments, the unpackaged pciAAV genome described herein comprises a positive single-stranded DNA sequence of the GOI, for example, between segment (a) and segment (c) or between segment (c) and segment (e). In some embodiments, the unpackaged pciAAV genome comprises a negative single-stranded DNA sequence of the GOI, for example, between segment (a) and segment (c) or between segment (c) and segment (e). In some embodiments, the unpackaged pciAAV genome comprises both a positive single-stranded DNA sequence and a negative single-stranded DNA sequence of the GOI, for example, between segment (a) and segment (c) and/or between segment (c) and segment (e).

In some embodiments, the unpackaged pciAAV genome comprises a positive or negative single-stranded DNA sequence of the GOI between segment (a) and segment (c) and a positive or negative single-stranded DNA sequence of the GOI between segment (c) and segment (e).

In some embodiments, the nucleotide sequence encoding the GOI comprises a forward GOI expression cassette. In some exemplary embodiments, the forward GOI expression cassette may comprise, in a 5′-3′ direction, a promoter, a GOI open reading frame (ORF), and a poly(A) sequence. In some exemplary embodiments, the forward GOI expression cassette may further comprise an enhancer or intron. In some exemplary embodiments, the enhancer or intron is located between the promoter and the GOI open reading frame. In some exemplary embodiments, the forward GOI expression cassette may comprise, in a 5′-3′ direction, DNA sequences for gene editing or gene regulation. In some exemplary embodiments, filler sequences may be included, for example, upstream of the promoter and/or downstream of the poly(A) sequence.

In some embodiments, the nucleotide sequence encoding the GOI comprises a reverse GOI expression cassette. In some exemplary embodiments, the reverse GOI expression cassette may comprise, in a 5′-3′ direction, the reverse complement sequence of the poly(A) sequence, the reverse complement sequence of the GOI open reading frame, and the reverse complement sequence of the promoter. In some exemplary embodiments, the reverse GOI expression cassette may further comprise an enhancer or intron. In some exemplary embodiments, the enhancer or intron is located between the reverse complement sequence of the promoter and the reverse complement sequence of the GOI open reading frame. In some exemplary embodiments, the reverse GOI expression cassette may comprise, in a 5′-3′ direction, the reverse complement sequence of DNA sequences for gene editing or gene regulation. In some exemplary embodiments, filler sequences may be included, for example, downstream of the reverse promoter and/or upstream of the reverse poly(A) sequence.

In some embodiments, the promoter may include, for example, a CMV promoter, a CAG promoter, or a cell-specific promoter such as a GFAP promoter or an hAAT promoter.

In some embodiments, the unpackaged pciAAV genome does not comprise nucleotide sequences encoding the rep gene and/or cap gene.

As used herein, the term “protection sequence” or “protection DNA sequence” refers to DNA sequences that are designed and constructed at the position of impurity DNA to prevent impurity DNA from being packaged into the rAAV capsid, wherein such DNA does not harm cells.

In some embodiments, the unpackaged pciAAV genome may be constructed in a plasmid (e.g., a pciAAV transgenic plasmid). The pciAAV transgenic plasmids described herein may include any plasmid suitable for producing the unpackaged pciAAV genome described herein. Suitable pciAAV transgenic plasmids may be based on, for example, but not limited to, pFastBacdual, pFastBacl plasmids, etc.

The unpackaged pciAAV genome may subsequently be packaged into capsid proteins using a pciAAV vector packaging system to form a pciAAV vector. In some embodiments, the pciAAV vector packaging system may include, for example, an insect cell-baculovirus packaging system, a mammalian cell packaging system, etc.

pciAAV Transgenic Plasmid

In another aspect, provided herein is a pciAAV transgenic plasmid comprising the unpackaged pciAAV genome described herein.

The pciAAV transgenic plasmids described herein may include any plasmid suitable for producing the unpackaged pciAAV genome described herein. Suitable pciAAV transgenic plasmids may be based on, for example but not limited to, pFastBacdual plasmid, pFastBacl plasmid, and the like.

In some exemplary embodiments, the pciAAV transgenic plasmid may be designed to encode a positive single-stranded DNA sequence of the GOI, as described above. In some exemplary embodiments, the pciAAV transgenic plasmid may be designed to encode a negative single-stranded DNA sequence of the GOI, as described above.

The pciAAV transgenic plasmid may be packaged, along with Rep and Cap gene expression plasmids, through a pciAAV vector packaging system to produce the pciAAV vector described herein.

pciAAV Vector

In another aspect, provided herein is a pciAAV vector comprising:
a capsid protein; and
a pciAAV genome packaged by the capsid protein;
wherein the pciAAV genome packaged by the capsid protein is derived from the unpackaged pciAAV genome described herein.

In some embodiments, the pciAAV genome packaged by the capsid protein is derived from segments (a)-(c) or segments (c)-(e) of the unpackaged pciAAV genome described herein.

As used herein, the term “pciAAV vector,” also referred to as “pciAAV viral particle,” is produced by packaging the unpackaged pciAAV genome into AAV capsid proteins (e.g., using a pciAAV vector packaging system).

In some embodiments, examples of the pciAAV vector packaging system may include, for example, an insect cell-baculovirus packaging system, a mammalian cell packaging system, and the like.

In some embodiments, the pciAAV genome packaged by the capsid protein (also referred to as the “packaged pciAAV genome”) is single-polar and single-stranded DNA. As used herein, the term “single-polar” refers to a single DNA polarity, meaning that the pciAAV vector either contains positive strand DNA or negative strand DNA, but not both in the same pciAAV vector.

In some embodiments, the packaged pciAAV genome forms hairpin structures at both ends (e.g., with a complete ITR at one end and a modified ITR at the other end).

In some embodiments, the packaged pciAAV genome does not comprise nucleotide sequences encoding the rep gene and/or cap gene.

In some embodiments, the packaged pciAAV genome comprises a positive single-stranded DNA sequence and/or a negative single-stranded DNA sequence of the GOI. In some embodiments, the packaged pciAAV genome comprises a positive single-stranded DNA sequence of the GOI. In some embodiments, the packaged pciAAV genome comprises a negative single-stranded DNA sequence of the GOI.

In some embodiments, the nucleotide sequence encoding the GOI comprises a forward GOI expression cassette. In some exemplary embodiments, the forward GOI expression cassette may comprise, in a 5′-3′ direction, a promoter, a GOI open reading frame (ORF), and a poly(A) sequence. In some exemplary embodiments, the forward GOI expression cassette may further comprise an enhancer or intron. In some exemplary embodiments, the enhancer or intron is located between the promoter and the GOI open reading frame. In some exemplary embodiments, the forward GOI expression cassette may comprise, in a 5′-3′ direction, DNA sequences for gene editing or gene regulation.

In some embodiments, the nucleotide sequence encoding the GOI comprises a reverse GOI expression cassette. In some exemplary embodiments, the reverse GOI expression cassette may comprise, in a 5′-3′ direction, the reverse complement sequence of the poly(A) sequence, the reverse complement sequence of the GOI open reading frame, and the reverse complement sequence of the promoter. In some exemplary embodiments, the reverse GOI expression cassette may further comprise an enhancer or intron. In some exemplary embodiments, the enhancer or intron is located between the reverse complement sequence of the promoter and the reverse complement sequence of the GOI open reading frame. In some exemplary embodiments, the reverse GOI expression cassette may comprise, in a 5′-3′ direction, the reverse complement sequence of DNA sequences for gene editing or gene regulation.

In some embodiments, the promoter may include, for example, a CMV promoter, a CAG promoter, or a cell-specific promoter such as a GFAP promoter, an hAAT promoter, and the like.

In some embodiments, the pciAAV vector is a group of pciAAV vectors containing at least a first pciAAV vector and a second pciAAV vector, wherein each of the first and second pciAAV vectors has a complete ITR at one end and a modified ITR at the other end. In some embodiments, the pciAAV genome of the first pciAAV vector is derived from segments (a)-(c) or segments (c)-(e) of the unpackaged pciAAV genome described herein. In some embodiments, the pciAAV genome of the second pciAAV vector is derived from segments (a)-(c) or segments (c)-(e) of the unpackaged pciAAV genome described herein.

In some embodiments, the GOI includes filler sequences at one end (e.g., the 5′ or 3′ end) or both ends (e.g., the 5′ and 3′ ends). In some embodiments, filler sequences are included between the modified ITR (e.g., segment (a) or (e)) and the complete ITR (e.g., segment (c)). “Filler sequences” generally refer to nucleotide sequences contained in a larger nucleic acid molecule (e.g., a plasmid vector), typically used to create a desired spacing between two nucleic acid elements (e.g., between a promoter and a coding sequence such as the GOI) or to extend the nucleic acid molecule to a desired length. Filler sequences do not contain protein-coding information and may have an unknown or synthetic origin and/or be unrelated to other nucleic acid sequences within the larger nucleic acid molecule.

In some embodiments, the length of the packaged pciAAV genome is approximately 4.7 kb (e.g., at least 4.0 kb, at least 4.1 kb, at least 4.2 kb, at least 4.3 kb, at least 4.5 kb, at least 4.6 kb, or up to 4.7 kb).

In some embodiments, the pciAAV vector comprises either a positive single-stranded DNA sequence of the GOI or a negative single-stranded DNA sequence of the GOI.

In some embodiments, the capsid protein is an AAV capsid protein. In some embodiments, the capsid protein may be selected from capsid proteins of various AAVs comprising AAV1 through AAV12 (AAV1, AAV2, AAV3, AAV4, AAV5, AAV6, AAV7, AAV8, AAV9, AAV10, AAV11, AAV12).

In some embodiments, the capsid protein is an AAV capsid protein variant, such as a tyrosine single/multi-amino acid variant, a tyrosine-serine-threonine variant, a multiseroype chimera such as AAV-DJ, or a polypeptide-inserted variant, among others.

In some embodiments, the capsid protein may be produced using a pciAAV vector packaging system with a plasmid expressing the AAV capsid protein Cap.

In some embodiments, the pciAAV vector may be produced using a pciAAV transgenic plasmid (producing the unpackaged pciAAV genome) and plasmids expressing the AAV capsid protein Cap and the replication protein Rep (producing Cap and Rep proteins), with a pciAAV vector packaging system. In some embodiments, the AAV Cap-encoding gene and the AAV Rep-encoding gene may reside on the same plasmid or on two different plasmids.

In some embodiments, the AAV Cap and/or AAV Rep expression plasmids may include Cap and/or Rep gene expression cassettes and required expression elements, and intron sequences for enhancing expression. There are no specific limitations on the plasmids used for expressing AAV Cap and/or Rep; those skilled in the art may routinely use AAV Cap and/or Rep expression plasmids. See, for example, Urabe M, Ding C, Kotin RM. Insect cells as a factory to produce adeno-associated virus type 2 vectors. Hum Gene Ther. 2002 Nov. 1; 13(16):1935-43. doi: 10.1089/10430340260355347. PMID: 12427305.

Method for Packaging pciAAV Vector

In another aspect, provided herein is a method for packaging a pciAAV vector, comprising: transforming DH10Bac competent E. coli cells with the pciAAV transgenic plasmid described herein and Cap and Rep expression plasmids, respectively; performing at least one round of blue-white colony screening, picking white colonies, amplifying, and extracting recombinant bacmids; transfecting insect cells with the recombinant bacmids to produce recombinant baculovirus; and extracting the recombinant baculovirus and infecting insect cells with the recombinant baculovirus to obtain the pciAAV vector.

In some embodiments, the competent E. coli cells are DH10Bac competent E. coli cells.

In some embodiments, the pciAAV vector packaging system is an insect cell-baculovirus packaging system.

In some embodiments, at least one round of blue-white colony screening is performed, white colonies are picked, amplified, and recombinant bacmids are extracted. In some embodiments, two or more rounds of blue-white colony screening are performed, white colonies are picked, amplified, and recombinant bacmids are extracted.

In some embodiments, the recombinant bacmids are transfected into insect cells (e.g., Sf9 insect cells) to produce recombinant baculovirus (e.g., P3 generation recombinant baculovirus).

In some embodiments, recombinant baculovirus (e.g., two or three types of P3 generation recombinant baculovirus) is used to infect (e.g., co-infect) insect cells (e.g., Sf9 insect cells) to package and obtain the pciAAV vector.

FIG. 2 provides a schematic diagram of the construction and packaging of a pciAAV vector according to an exemplary embodiment described herein. In the unpackaged pciAAV genome to be packaged by the pciAAV vector, the GOI (positive or negative strand) to be packaged has a complete ITR upstream and a modified ITR lacking a D element and a trs sequence downstream. An additional 4.4 kb DNA sequence containing the GOI is added upstream of the above segments, which itself has a modified ITR lacking a D element and a trs sequence upstream. The resulting AAV gene vector only contains DNA molecules of the GOI (positive or negative strand) and does not contain plasmid backbone DNA impurities.

The method for packaging rAAV vectors using an insect cell-baculovirus packaging system may also refer to, for example, Urabe M, Ding C, Kotin RM. Insect cells as a factory to produce adeno-associated virus type 2 vectors. Hum Gene Ther. 2002 Nov. 1; 13(16):1935-43. doi: 10.1089/10430340260355347. PMID: 12427305.

Applications and Method for Using pciAAV

In another aspect, provided herein is a method for delivering a GOI to cells, comprising contacting the cells with one or more pciAAV vectors described herein; wherein the genome of the one or more pciAAV vectors comprises a gene expression cassette for the GOI and/or optionally other DNA sequences; wherein the pciAAV vector is derived from packaging the unpackaged pciAAV genome described herein.

In some embodiments, the cells may be eukaryotic cells. In some embodiments, the cells may be animal cells. In some embodiments, the cells may be vertebrate cells. In some embodiments, the cells may be mammalian cells, such as human cells.

In some embodiments, the genome of the pciAAV vector comprises a positive single-stranded DNA sequence or a negative single-stranded DNA sequence of the GOI. In some embodiments, one or more pciAAV vectors comprise pciAAV vectors containing only the positive single-stranded DNA sequence of the GOI and/or pciAAV vectors containing only the negative single-stranded DNA sequence of the GOI.

In some embodiments, one or more pciAAV vectors comprise two or more pciAAV vectors, including at least a first pciAAV vector containing the positive single-stranded DNA sequence of the GOI and a second pciAAV vector containing the negative single-stranded DNA sequence of the GOI.

In some embodiments, cells are contacted with a pciAAV vector containing the positive single-stranded DNA sequence of the GOI. In some embodiments, cells are contacted with a pciAAV vector containing the negative single-stranded DNA sequence of the GOI. In a preferred embodiment, cells are contacted with both a first pciAAV vector containing the positive single-stranded DNA sequence of the GOI and a second pciAAV vector containing the negative single-stranded DNA sequence of the GOI. In some embodiments, cells are simultaneously contacted with the first pciAAV vector containing the positive single-stranded DNA sequence of the GOI and the second pciAAV vector containing the negative single-stranded DNA sequence of the GOI. In some embodiments, cells are sequentially contacted (e.g., within 24 hours) with the first pciAAV vector containing the positive single-stranded DNA sequence of the GOI and the second pciAAV vector containing the negative single-stranded DNA sequence of the GOI, or vice versa. It should be understood that the terms “first pciAAV vector” and “second pciAAV vector” are used herein only to distinguish the two and do not imply any restriction on the order of use.

When cells are infected with a pciAAV vector containing the positive single-stranded DNA sequence of the GOI or a pciAAV vector containing the negative single-stranded DNA sequence of the GOI, the 3′ end may serve as a primer to synthesize the complementary DNA strand and initiate gene expression.

By co-using a pciAAV vector containing the positive single-stranded DNA sequence of the GOI and a pciAAV vector containing the negative single-stranded DNA sequence of the GOI, the positive and negative strands of the pciAAV genome can rapidly complement each other in the host cell nucleus to form double-stranded molecules, thereby initiating gene expression.

In another aspect, provided herein is an isolated host cell comprising one or more pciAAV vectors described herein.

In some embodiments, the host cell may be a eukaryotic cell. In some embodiments, the host cell may be an animal cell. In some embodiments, the host cell may be a vertebrate cell. In some embodiments, the host cell may be a mammalian cell. In some embodiments, the host cell may be a human cell.

In some embodiments, the host cell is obtained by contacting a eukaryotic cell (e.g., an animal cell, such as a mammalian cell or human cell) with one or more pciAAV vectors described herein.

In another aspect, provided herein is one or more pciAAV vectors described herein for use in gene expression. Also provided is the use of one or more pciAAV vectors described herein in the preparation of products for gene expression. In some embodiments, the subject of gene expression is an animal, specifically a vertebrate, more specifically a mammal, and even more specifically a human.

In another aspect, provided herein is one or more pciAAV vectors described herein for use in gene therapy. Also provided is the use of one or more pciAAV vectors described herein in the preparation of products for gene therapy. In some embodiments, the subject of gene therapy is an animal, specifically a vertebrate, more specifically a mammal, and even more specifically a human.

In another aspect, provided herein is one or more pciAAV vectors described herein for use in gene editing. Also provided is the use of one or more pciAAV vectors described herein in the preparation of products for gene editing. In some embodiments, the subject of gene editing is an animal, specifically a vertebrate, more specifically a mammal, and even more specifically a human.

In another aspect, provided herein is one or more pciAAV vectors described herein for use in gene regulation. Also provided is the use of one or more pciAAV vectors described herein in the preparation of products for gene regulation. In some embodiments, the subject of gene regulation is an animal, specifically a vertebrate, more specifically a mammal, and even more specifically a human.

### EXAMPLES

The invention is further explained with the following specific examples. It should be understood that these examples are intended only to illustrate the invention and not to limit its scope. Those skilled in the art can make appropriate modifications and changes, all of which fall within the scope of the invention.

Construction of Plasmid Vectors for pciAAV Vector Packaging

Plasmid 1: pFBD-ITR-CMV-EGFP-PolyA-1.9kb stuff DNA-ITR, used for packaging a standard rAAV-CMV-EGFP-PolyA vector.

Plasmid 1 was constructed as follows:
Primer design and use:
Primer 1: 5′-cacgtgcggaccgagtgcatggtccggatgccca-3′ (SEQ ID NO: 1, synthesized by Sangon Biotech, Shanghai, China);
Primer 2: 5′-gccgctcggtccgagggtacaaggcagggcctgc-3′ (SEQ ID NO: 2, synthesized by Sangon Biotech, Shanghai, China).

A 1.9 kb stuff DNA fragment was PCR-amplified, digested with RsrII to generate sticky ends, and inserted into an RsrII-digested and CIAP-treated pFastBacdual-ITR-EGFP plasmid vector.

(The construction of pFastBacdual-ITR-EGFP plasmid refers to the method described in: Tai-Ming Li et al., Preparation of AAV-ITR Gene Expression Microcarrier Using Insect Cells, Chinese Journal of Biotechnology, 2015, 31(8):1232, section 1.2.1, “Construction of pFastBacdual-ITR-EGFP plasmid”).

Plasmid 2: pFB1-ΔDtrsITR-1.9kb stuff DNA-CMV-EGFP-PolyA-ITR-1.9kb stuff DNA-CMV-EGFP-PolyA-ΔDtrsITR, used for packaging pciAAV-CMV-EGFP-PolyA vector.

Plasmid 2 was constructed as follows:
Step 1: Construction of pFB1-ΔDtrsITR-CMV-EGFP-PolyA-ITR-ANN.

Primer design and use:
Primer 3: 5′-agcaccagtcgcggccgctttagatccgaaccagataag-3′ (SEQ ID NO: 3, synthesized by Sangon Biotech, Shanghai, China);
Primer 4: 5′-ggccaacctaggagggctgctagcaccagtcgcggccgcttt-3′ (SEQ ID NO: 4, synthesized by Sangon Biotech, Shanghai, China);
Primer 5: 5′-gggaaagccggcgaacgtggcgagaaaggaagg-3′ (SEQ ID NO: 5, synthesized by Sangon Biotech, Shanghai, China).

A PCR product containing AvrII/NheI/NotI restriction sites (ANN)-vector-NgoMIV was generated in two rounds of PCR amplification. The AvrII/NgoMIV-digested ANN-vector-NgoMIV fragment with sticky ends was inserted into an AvrII/NgoMIV-digested pFB1-ΔDtrsITR-CMV-EGFP-PolyA-ITR vector, introducing three restriction sites (AvrII/NheI/NotI).

Step 2: Construction of pFB1-ΔDtrsITR-1.9kb stuff DNA-CMV-EGFP-PolyA-ITR-ANN.

Primer design and use:
Primer 6: 5′-tagtcgacgcgtagtgcatggtccggatgccca-3′ (SEQ ID NO: 6, synthesized by Sangon Biotech, Shanghai, China);
Primer 7: 5′-aactagacgcgtagggtacaaggcagggcctgc-3′ (SEQ ID NO: 7, synthesized by Sangon Biotech, Shanghai, China).

A 1.9 kb stuff DNA fragment was PCR-amplified, digested with MluI to generate sticky ends, and inserted into an MluI-digested and CIAP-treated pFB1-ΔDtrsITR-CMV-EGFP-PolyA-ITR-ANN vector.

Step 3: Construction of pFB1-ΔDtrsITR-1.9kb stuff DNA-CMV-EGFP-PolyA-ITR-CMV-EGFP-PolyA.

Primer design and use:
Primer 8: 5′-tttgtagctagcctagttattaatagtaatcaa-3′ (SEQ ID NO: 8, synthesized by Sangon Biotech, Shanghai, China);
Primer 9: 5′-tctaaagcggccgctacaaaatcagaaggacaggga-3′ (SEQ ID NO: 9, synthesized by Sangon Biotech, Shanghai, China).

The CMV-EGFP-PolyA fragment was PCR-amplified, digested with NheI/NotI to generate sticky ends, and inserted into an NheI/NotI-digested pFB1-ΔDtrsITR-1.9kb stuff DNA-CMV-EGFP-PolyA-ITR-ANN vector.

Step 4: Construction of pFB1-ΔDtrsITR-1.9kb stuff DNA-CMV-EGFP-PolyA-ITR-1.9kb stuff DNA-CMV-EGFP-PolyA.

Primer design and use:
Primer 10: 5′-agggctgctagcagtgcatggtccggatgccca-3′ (SEQ ID NO: 10, synthesized by Sangon Biotech, Shanghai, China);
Primer 11: 5′-aactaggctagcagggtacaaggcagggcctgc-3′ (SEQ ID NO: 11, synthesized by Sangon Biotech, Shanghai, China).

A 1.9 kb stuff DNA fragment was PCR-amplified, digested with NheI to generate sticky ends, and inserted into an NheI-digested and CIAP-treated pFB1-ΔDtrsITR-1.9kb stuff DNA-CMV-EGFP-PolyA-ITR-CMV-EGFP-PolyA vector.

Step 5: Construction of pFB1-ΔDtrsITR-1.9kb stuff DNA-CMV-EGFP-PolyA-ITR-1.9kb stuff DNA-CMV-EGFP-PolyA-ΔDtrsITR.

Primer design and use:
Primer 12: 5′-tttgtagcggccgcattcttctagagctccatggt-3′ (SEQ ID NO: 12, synthesized by Sangon Biotech, Shanghai, China);
Primer 13: 5′-tctaaagcggccgcccggaatattaatagccgcgg-3′ (SEQ ID NO: 13, synthesized by Sangon Biotech, Shanghai, China).

The ΔDtrsITR fragment was PCR-amplified, digested with NotI to generate sticky ends, and inserted into a NotI-digested and CIAP-treated pFB1-ΔDtrsITR-1.9kb stuff DNA-CMV-EGFP-PolyA-ITR-1.9kb stuff DNA-CMV-EGFP-PolyA vector.

### Preparation of Recombinant Bacmid

The recombinant plasmids constructed in the previous step were used to prepare recombinant Bacmids, by the following process:
1. 100 μL of DH10Bac competent cells were slowly thawed on ice for 1-3 minutes.
2. 500 ng of plasmid DNA was added, and the mixture was gently mixed.
3. The mixture was placed on ice for 30 minutes, heat shocked at 42° C. for 90 seconds, and immediately returned to ice for 3 minutes.
4. 890 μL of LB medium was added, and the mixture was shaken at 37° C. and 225 rpm for 2-3 hours.
5. On a pre-prepared 90 mm KTG-resistant agar LB solid medium plate containing final concentrations of 50 g/mL kanamycin (Kan), 77 g/mL gentamicin (Gen), and 10 g/mL tetracycline (Tet), 40 μL of 2% (20 mg/mL) X-gal and 20 μL of 20% (200 mg/mL) IPTG were added to the center. A sterile spreader was used to evenly distribute the mixture over the plate surface, and the plate was incubated in an oven until all liquid had evaporated.
6. Bacterial suspensions were spread in gradients of 100 μL and 300 μL onto the KTG-resistant solid agar plates.
7. The plates were incubated at 37° C. for 48 hours. Two white colonies were picked and streaked onto new KTG-resistant solid agar plates, which were incubated overnight at 37° C.
8. Two single colonies were picked, and PCR identification was performed. The Bacmid was identified using PCR with primers specific for the target gene (F/R) and PUC M13 (F/R).
9. The correctly identified Bacmid colonies were inoculated into 10 mL of LB medium containing Kan, Gen, and Tet. The culture was shaken for 16-18 hours. Recombinant Bacmid DNA was extracted and isolated using an OMEGA kit according to the manufacturer's instructions. The Bacmid DNA concentration was measured, aliquoted, and stored at −20° C.

### Preparation of Baculovirus

### 1. Preparation of Transfection Reagents

Preparation of 1×HBS (200 mL):

| | | |
|---|---|---|
| Hepes: | 0.954 | g |
| NaCl: | 1.754 | g |
| Sterile ddH2O: | 150 | mL |

The pH was adjusted to 7.4 using 1M NaOH.

The solution was made up to 200 mL, filter sterilized in a biosafety cabinet, and stored at 4° C.

Preparation of PEI (20 mL):

| | | |
|---|---|---|
| PEI: | 0.043 | g |
| Anhydrous ethanol: | 1 | mL |

PEI was dissolved thoroughly, then made up to 20 mL with 1×HBS. The solution was freeze-thawed three times (−20° C. for freezing, room temperature for thawing) and stored at −20° C.

### 2. Cell Plating

A 6-well plate was used, and suspension-cultured cells were pipetted and counted to achieve a cell density of 2×10⁶ cells/mL. 2 mL of cells was added to each well, ensuring a cell viability of above 95%.

3. Transfection (per well)

Solution A: PEI: 6 μL PEI and 94 μL 1×HBS were mixed, and the solution was allowed to stand for 4 minutes.

Solution B: 3 g of Bacmid DNA (pre-inactivated at 65° C. for 30 minutes) was mixed with 1×HBS to a final volume of 100 μL. The solution was mixed gently.

100 μL of Solution A was added to Solution B, mixed well, and incubated at room temperature for 30 minutes. The mixture was then added to the wells with plated cells and cultured for 96 hours.

### 4. Virus Amplification

### 1) Isolation of P1:

After confirming that the cells were in the late infection stage (96 h), 2 mL of virus-containing supernatant was collected from each well into a sterile 15 mL centrifuge tube. The supernatant was centrifuged at 500 g for 5 minutes to remove cell debris.

The supernatant was transferred to a sterile EP tube and stored at 4° C. in dark place. For long-term storage, the solution was aliquoted and frozen at −80° C.

### 2) Virus Amplification to Obtain P2:

8 mL of suspension-cultured cells at an MOI of 0.1 at a density of 2×10⁶ cells/mL were taken. The required P1 volume was calculated as 1.6 mL. The mixture was incubated at 27° C. for 72 hours. Suspension-cultured cells were collected into a sterile 15 mL centrifuge tube and centrifuged at 500 g for 5 minutes. The supernatant was aliquoted into sterile EP tubes as P2, stored at 4° C. in dark place, or aliquoted and frozen at −80° C. for long-term storage.

### 3) Virus Amplification to Obtain P3:

P3 was obtained using the same method. 10 mL of suspension-cultured cells at an MOI of 0.1 at a density of 2×10⁶ cells/mL were mixed with 200 μL of P2 stock solution. The culture was incubated for 72 hours, and P3 was collected.

Typically, the P1 virus titer ranged between 1×10⁶-1×10⁷, and the P2 titer ranged between 1×10⁷-1×10⁸.

### 4) Virus Packaging:

100 mL of suspension-cultured cells at an MOI of 1 at a density of 5×10⁶ cells/mL were used. 5 mL each of P3 Rep, Cap, and target gene were added. The culture was incubated for 72 hours, and the cells were collected.

Packaging of pciAAV Vectors

The pciAAV vector packaging system described herein can be an insect cell baculovirus packaging system or a mammalian cell packaging system. The insect cells used are Sf9 cells. First, one or two recombinant plasmids expressing AAV replication protein Rep and AAV capsid protein Cap (with the Rep and Cap genes either on the same plasmid or on two separate plasmids) and another plasmid containing the pciAAV DNA genome were transformed into DH10Bac competent E. coli cells. After two rounds of blue-white colony screening, colonies containing recombinant bacmids appeared white, while non-recombinant colonies appeared blue. White colonies were picked, amplified, and recombinant bacmids were extracted.

Then, using insect cell transfection reagents, the above two or three recombinant bacmids were transfected into Sf9 insect cells. 4 to 5 days later, the cell supernatant was collected to obtain the P1 generation of recombinant baculoviruses from insect cells. The P1 generation of recombinant baculoviruses was further amplified through two rounds of infection in Sf9 insect cells to obtain the P3 generation of recombinant baculoviruses. The titer of the P3 baculovirus was determined using a plaque assay, with the virus titer (pfu/mL) calculated as 1/dilution factor x number of plaques x 1/seeding volume per well.

Finally, the two or three P3 recombinant baculoviruses were co-infected into Sf9 insect cells to package and produce pciAAV vector virus particles. Detailed procedures can refer to the publication: Urabe M, Ding C, Kotin RM. Insect cells as a factory to produce adeno-associated virus type 2 vectors. Hum Gene Ther. 2002 Nov. 1; 13(16):1935-43. doi:10.1089/10430340260355347. PMID:12427305.

A schematic diagram of the insect cell baculovirus packaging system is shown in FIG. 2.

### Iodixanol Density Gradient Ultracentrifugation Purification

### 1. Obtaining Cell Lysate

1) Cells were collected by centrifuging at 1000 g for 5 minutes after 72 hours of co-transfection.
2) The supernatant was discarded, and the cells were resuspended in 10 mL of Lysis Buffer.

Lysis Buffer (1 μL):

| | | |
|---|---|---|
| NaCl: | 8.766 | g |
| Tris: | 6.055 | g |
| ddH₂O: | 950 | mL |

The pH was adjusted to 8.5 using 5M HCl, and the buffer was made up to 1 μL. The buffer was filter-sterilized in a biosafety cabinet and stored at 4° C.
3) The suspension was subjected to repeated freeze-thaw cycles (3-4 times) using liquid nitrogen and thawing at 37° C. until the solution became clear.
4) The supernatant was collected by centrifuging at 5000 g for 30 minutes at 4° C. The pellet was resuspended in 4-5 mL PBS, and the supernatant was collected again by centrifugation.
5) DNase (10 L/mL) and RNase (1 L/mL) were added to the supernatant, followed by digestion in a 37° C. water bath for 1 hour in preparation for purification.

### 2. Iodixanol Density Gradient Ultracentrifugation Purification of Virus

1) A 60% iodixanol solution was diluted with PBS-MK (1×PBS, 1 mM MgCl₂, 2.5 mM KCl) to prepare 15%, 25%, 40%, and 58% iodixanol solutions. Solid NaCl was added to the 15% solution to achieve a final concentration of 1 M.
2) Phenol red solution (0.5%) was added to the 15% and 25% solutions at a final concentration of 1.5 L/mL, and to the 58% solution at a final concentration of 0.5 L/mL.
3) Using a 1.27×120 mm flat spinal needle, 8 mL of the 15% iodixanol solution, 6 mL of the 25% iodixanol solution, 8 mL of the 40% iodixanol solution, and 5 mL of the 58% iodixanol solution were sequentially added from the bottom of a 39 mL quick-seal centrifuge tube, avoiding the formation of air bubbles.
4) The cell lysate was carefully layered onto the iodixanol density gradient solution to fill the tube up to the rim. If necessary, Lysis Buffer was added to top off the volume. The tubes were balanced and centrifuged at 69,000 rpm at 18° C. for 1.5 hours using a BECKMAN COULTER centrifuge with a 70Ti rotor.
5) The bottom of the quick-seal tube was pierced, and the initial 4 mL (corresponding to the 58% phase) was discarded. A total of 6 mL of the solution from the tube was collected.
6) The solution was concentrated using an ultrafiltration device (Amicon Ultra-4 Centrifugal Filters, Ultracel-100k) by centrifuging at 3000 g for 5 minutes. The concentrate was washed repeatedly with PBS (1 ×PBS, supplemented with 5M NaCl to a final concentration of 350 mM) until the residual iodixanol concentration was <0.1%. The virual particles were concentrated to approximately 200 μL per 100 mL of packaging volume.
7) Glycerol was added to a final concentration of 5%. The solution was sterilized by filtration, aliquoted, and stored at −80° C.
   Method for Quantitative Fluorescence PCR Detection of pciAAV Vector Titer RT-PCR Method
1. The ITR-CMV-EGFP-PolyA-ITR plasmid was diluted 10-fold and further diluted in a gradient across five steps to prepare the standard curve.
2. The virus was diluted 10-fold and further diluted in a gradient across four steps.
3. The buffer was prepared (protected from light) as follows:
   2×SuperpreMix Plus (SYBR Green): 10 μL

Primer 14,
5′-TCCGCGTTACATAACTTACGG-3′
(SEQ ID NO: 14; synthesized by Sangon Biotech, Shanghai): 0.3 μL
Primer 15,
5′-GGGCGTACTTGGCATATGAT-3′
(SEQ ID NO: 15; synthesized by Sangon Biotech, Shanghai): 0.3 μL

ddH₂0: 4.4 μL
4. The reaction mixture was prepared as a 20 μL system: 5 μL template+15 μL Buffer. Each gradient was prepared in duplicate wells.
5. The RT-PCR (Roche LightCycler) program was set as follows:

| | | | | | |
|---|---|---|---|---|---|
| Stage 1 | Pre-denaturation | Reps: 1 | 95° C. | 600 | s |
| Stage 2 | Cycling reaction | Reps: 40 | 95° C. | 10 | s |
| | | | 55° C. | 30 | s |
| | | | 72° C. | 30 | s |
| Stage 3 | Melting curve | Reps: 1 | 95° C. | 15 | s |
| | | | 60° C. | 60 | s |
| | | | 95° C. | 15 | s |

SDS-PAGE Electrophoresis Analysis of pciAAV Vector Capsid Proteins

### 1. Gel Preparation

### 1) Separating Gel (10%):

The following components (total volume 9.093 mL) were added into a preparation beaker for separating gel: double-distilled water (3.69 mL), 30% acrylamide gel solution (acrylamide:bis-acrylamide=29:1, 2.97 mL), 2 M Tris, pH 8.8 (2.25 mL), 10% ammonium persulfate (90 μL), 10% SDS (90 μL), and TEMED (3 μL). The solution was mixed well and pipetted between two glass plates until the liquid level reached 1 cm below the comb. 75% ethanol was gently layered on top with a pipette, and the gel was left to polymerize.

After polymerization, the ethanol layer was removed.

### 2) Stacking Gel (5%):

The following components (total volume 2.357 mL) were added into a preparation beaker for stacking gel: double-distilled water (1.83 mL), 30% acrylamide gel solution (acrylamide:bis-acrylamide=29:1, 0.39 mL), 0.5 M Tris, pH 6.8 (0.75 mL), 10% ammonium persulfate (30 μL), 10% SDS (30 μL), and TEMED (2 μL). The solution was mixed and immediately pipetted on top of the separating gel between the glass plates. The comb was gently inserted, and the gel was left to polymerize. Once solidified, the gel plate was complete.

### 2. Sample Preparation

The virus samples were prepared using the same procedures for construction, packaging, and purification of the pciAAV vector described above.

Virus samples were mixed with 5× loading buffer in a 2:1 ratio, incubated in a boiling water bath for 10 minutes, and centrifuged briefly. The entire sample was used for electrophoresis.

### 3. Electrophoresis

1) The gel plate made of two glass plates were vertically mounted onto the power frame of the electrophoresis tank, ensuring the concave edge of the gel faced the power frame.
2) The gel plate and power frame were secured in the electrophoresis tank, and electrophoresis buffer was added as required. The comb was gently removed from the gel.
3) Using a micropipette, 15 μL of the prepared sample was carefully loaded into each well (sample loading sites) on the gel.
4) Electrophoresis was conducted with a voltage of 90 V for the stacking gel and 120 V for the separating gel. Electrophoresis was stopped when the bromophenol blue dye front reached approximately 1 cm above the bottom of the gel.

### 4. Staining

The gel plates were gently pried apart with a blade or thin plate, and the separating gel was carefully cut along the interface between the separating gel and the stacking gels. The separating gel was carefully transferred to a staining container, 100 mL of staining solution was added, and the container was covered and stained for 1-3 hours.

### 5. Destaining

The staining solution was discarded, and the gel was rinsed several times with water. The gel was placed in fresh water, just covering the gel surface, and microwaved at high power for 2 minutes. After microwaving, the gel was gently shaken. This process was repeated until the protein bands became clearly visible.

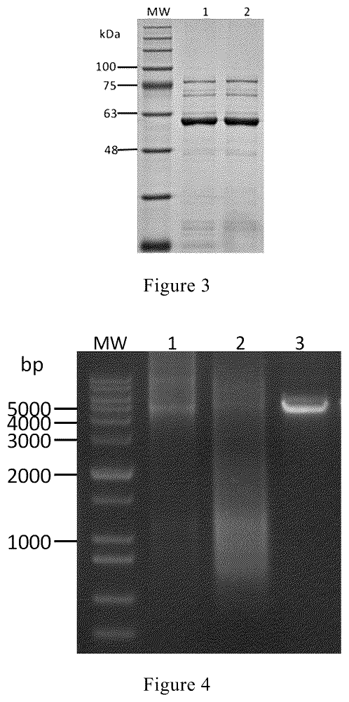

FIG. 3 shows the SDS-PAGE electrophoresis results of pciAAV vector capsid proteins. Lane 1 represents the pciAAV vector, and Lane 2 represents the rAAV vector. The composition of pciAAV capsid proteins is identical to that of standard rAAV capsid proteins, consisting of three proteins: VP1, VP2, and VP3, with an approximate ratio of 1:1:10.

Identification of the pciAAV Vector Genome

### DNA Neutral Agarose Gel Electrophoresis Analysis

1. An accurate amount of agarose powder and a measured volume of 1 × TAE electrophoresis buffer were added to an Erlenmeyer flask or glass bottle to prepare the agarose solution.
2. A Kimwipes paper was gently placed over the neck of the flask. For glass bottles, the cap was loosened. The suspension was heated in a boiling water bath or microwaved until the agarose was fully dissolved.
3. The clear solution was cooled to 40-50° C., and 4S red staining solution was added. The gel was quickly poured. After the gel had fully solidified, it was placed into the electrophoresis tank, and freshly prepared 1 × TAE electrophoresis buffer was added until the gel was just submerged.
4. The rAAV-CMV-EGFP-PolyA vector and pciAAV-CMV-EGFP-PolyA vector prepared as described above were used. Virus stock solutions (10 μL) were mixed with 1.1 L of 10× PCR Buffer and placed into a PCR machine under the following conditions: 95° C. for 5 minutes, 72° C. for 5 minutes, 55° C. for 5 minutes, 37° C. for 5 minutes, 80° C. for 5 minutes, 72° C. for 5 minutes, 55° C. for 5 minutes, and 37° C. for 15 minutes.
5. A 0.2× volume of 6× gel loading buffer was added to the denatured samples.
6. The entire volume of each sample mixed with 6× gel loading buffer was loaded into the wells. Electrophoresis was performed at 100 V for 50 minutes.
7. Imaging: The gel was placed in a gel imaging system for photographing and recording results.

FIG. 4 shows the results of neutral agarose gel electrophoresis of the pciAAV vector. Lane 1 represents the rAAV vector, in which the DNA primarily exists as 4.7 kb double-stranded DNA molecules formed by the complementary pairing of positive and negative DNA strands. Lane 2 represents the pciAAV vector, which carries two target gene DNA molecules: positive single-stranded DNA and negative single-stranded DNA (both approximately 4.7 kb in length). Due to the complementary nature of these DNA strands with opposite polarity, they appear as 4.7 kb double-stranded DNA molecules in neutral agarose gel electrophoresis, wherein the single polarity single-stranded DNA molecules, being smaller in molecular weight, are displayed as significantly smaller, often diffused DNA bands in the neutral gel. Lane 3 represents a 4.7 kb PCR DNA fragment.

### DNA Alkaline Agarose Gel Electrophoresis Analysis

### 1. Preparation of Alkaline Agarose Gel

Agarose powder (0.36 g) and 27 mL distilled water were added to an Erlenmeyer flask or glass bottle to prepare the agarose solution. A Kimwipes paper was gently placed over the neck of the Erlenmeyer flask. For glass bottles, the cap was loosened. The suspension was heated at medium power in a microwave oven until the agarose was fully dissolved. The clear solution was equilibrated in a 56° C. water bath for 5 minutes. Then, 3 mL of 10× alkaline gel electrophoresis buffer (equilibrated in a 56° C. water bath for 2 minutes) was added and mixed thoroughly. The gel was quickly poured. After the gel had fully solidified, it was placed in the electrophoresis tank, and freshly prepared 1× electrophoresis buffer (4° C.) was added until the gel was just submerged.

### 2. Sample Preparation

The rAAV-CMV-EGFP-PolyA vector and pciAAV-CMV-EGFP-PolyA vector prepared as described above were used. Virus samples (30 μL) were mixed with 3 μL of proteinase K (20 mg/mL) and digested at 65° C. for 15 minutes. The mixture was centrifuged at 12,000 g for 5 minutes, and 30 μL of the supernatant was collected. To this, 6 μL of 6× alkaline gel loading buffer was added.

### 3. Electrophoresis

The DNA samples were fully loaded into the sample wells. Electrophoresis was performed at a voltage of <3.5 V/cm (horizontal electrophoresis tank JY-SPAT, 27 V, for 3 hours).

### 4. Elution

The gel was placed in 400 mL of water for injection on a horizontal shaker at 56 rpm for 1 hour, with the water changed every 30 minutes.

### 5. Staining

The eluted gel was stained with 1× TAE staining solution containing 0.5 g/mL ethidium bromide (EB).

### 6. Imaging

The gel was placed in a gel imaging system for photographing and recording results.

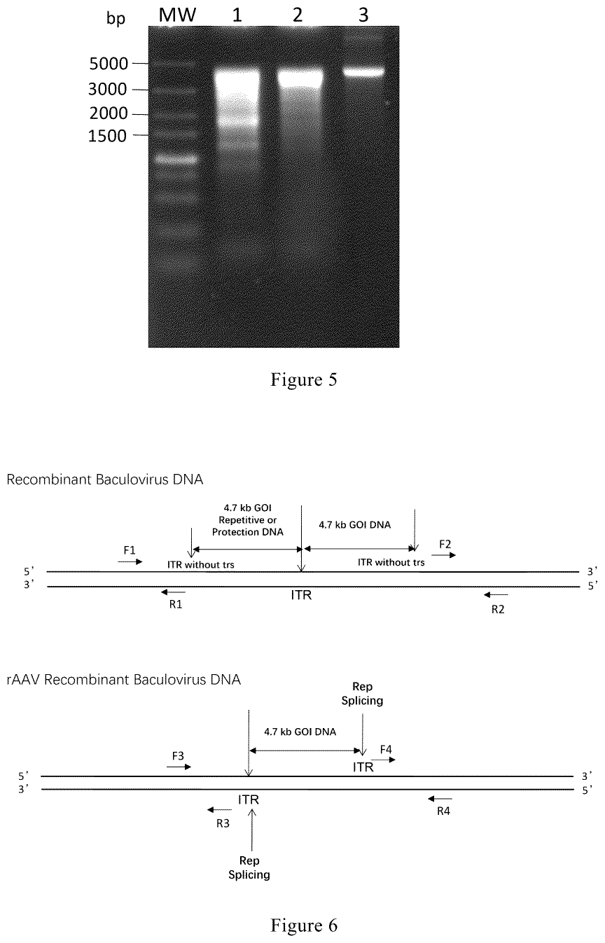

FIG. 5 shows the results of alkaline agarose gel electrophoresis of the pciAAV vector. Lane 1: The pciAAV vector. Under alkaline agarose gel electrophoresis analysis, pciAAV DNA typically appears as a single DNA band. The single-stranded DNA length is 4.7 kb, but due to potential premature breakage of DNA molecules during packaging, heterogeneous DNA lengths may be observed. Lane 2: The rAAV vector, in which the DNA mainly exists as 4.7 kb single-stranded DNA molecules. Lane 3: A 4.7 kb PCR DNA fragment.

PCR Analysis of Impurity DNA in pciAAV Vector

### 1. Preparation of Materials

Templates: pFBD-ITR-CMV-EGFP-PolyA-1.9kb stuff DNA-ITR, Bac-ITR-CMV-EGFP-PolyA-1.9kb stuff-ITR, rAAV2-ITR-CMV-EGFP-PolyA-1.9kb stuff-ITR.

pFB1-ΔDtrsITR-1.9kb stuff DNA-CMV-EGFP-PolyA-ITR-1.9kb stuff DNA-CMV-EGFP-PolyA-ΔDtrsITR, Bac-ΔDtrsITR-1.9kb stuff DNA-CMV-EGFP-PolyA-ITR-1.9kb stuff DNA-CMV-EGFP-PolyA-ΔDtrsITR, rAAV6-ΔDtrsITR-1.9kb stuff DNA-CMV-EGFP-PolyA-ITR-1.9kb stuff DNA-CMV-EGFP-PolyA-ΔDtrsITR (pciAAV-CMV-EGFP-PolyA vector virus).

### 2×Taq Master Mix (Nearshore Biology).

### 2. Primer Design for Verifying Impurity DNA

To verify whether plasmid impurity DNA fragments flank either side of the ΔDtrsITR in the rAAV6-ΔDtrsITR-1.9kb stuff DNA-CMV-EGFP-PolyA-ITR-1.9kb stuff DNA-CMV-EGFP-PolyA-ΔDtrsITR virus (pciAAV-CMV-EGFP-PolyA vector virus) packaged from pFB1-ΔDtrsITR-1.9kb stuff DNA-CMV-EGFP-PolyA-ITR-1.9kb stuff DNA-CMV-EGFP-PolyA-ΔDtrsITR, the following primers were designed for PCR amplification.

Primer sequences:

Primer 16:
ttaggtggcggtacttgggtc (SEQ ID NO: 16, synthesized by Sangon Biotech
[Shanghai] Co., Ltd.)
Primer 17:
cgcagcagggcagtcgcccta (SEQ ID NO: 17, synthesized by Sangon Biotech
[Shanghai] Co., Ltd.)
Primer 18:
tgattttgtagcggccgcatt (SEQ ID NO: 18, synthesized by Sangon Biotech
[Shanghai] Co., Ltd.)
Primer 19:
agcgtcgtaagctaatacgaa (SEQ ID NO: 19, synthesized by Sangon Biotech
[Shanghai] Co., Ltd.)
Primer 20:
atggtgagcaagggcgaggag (SEQ ID NO: 20, synthesized by Sangon Biotech
[Shanghai] Co., Ltd.)
Primer 21:
ttacttgtacagctcgtccat (SEQ ID NO: 21, synthesized by Sangon Biotech
[Shanghai] Co., Ltd.)

To verify whether plasmid impurity DNA fragments flank either side of the ITR in the rAAV2-ITR-CMV-EGFP-PolyA-1.9kb stuff-ITR virus (rAAV-CMV-EGFP-PolyA vector virus) packaged from pFBD-ITR-CMV-EGFP-PolyA-1.9kb stuff DNA-ITR, the following primers were designed for PCR amplification.

Primer sequences:

Primer 16:
ttaggtggcggtacttgggtc (SEQ ID NO: 16, synthesized by Sangon Biotech
[Shanghai] Co., Ltd.)
Primer 17:
cgcagcagggcagtcgcccta (SEQ ID NO: 17, synthesized by Sangon Biotech
[Shanghai] Co., Ltd.)
Primer 22:
gatcataatcagccatacca (SEQ ID NO: 22, synthesized by Sangon Biotech
[Shanghai] Co., Ltd.)
Primer 19:
agcgtcgtaagctaatacgaa (SEQ ID NO: 19, synthesized by Sangon Biotech
[Shanghai] Co., Ltd.)
Primer 20:
atggtgagcaagggcgaggag (SEQ ID NO: 20, synthesized by Sangon Biotech
[Shanghai] Co., Ltd.)
Primer 21:
ttacttgtacagctcgtccat (SEQ ID NO: 21, synthesized by Sangon Biotech
[Shanghai] Co., Ltd.)

### 3. PCR System (20 μL):

| Component | Volume (μL) |
|---|---|
| Template | 1 (1-10 ng) |
| 2 × Taq | 10 |
| H₂O | 8 |
| F Primer | 0.5 |
| R Primer | 0.5 |

### PCR Conditions:

| | |
|---|---|
| Pre-denaturation: | 95° C., 4 min |
| Denaturation: | 95° C., 30 sec |
| Annealing: | 56° C., 30 sec |
| Extension: | 72° C., 2 min |
| Final extension: | 72° C., 10 min |
| Cycles: | 25 |
| Hold: | 4° C. |

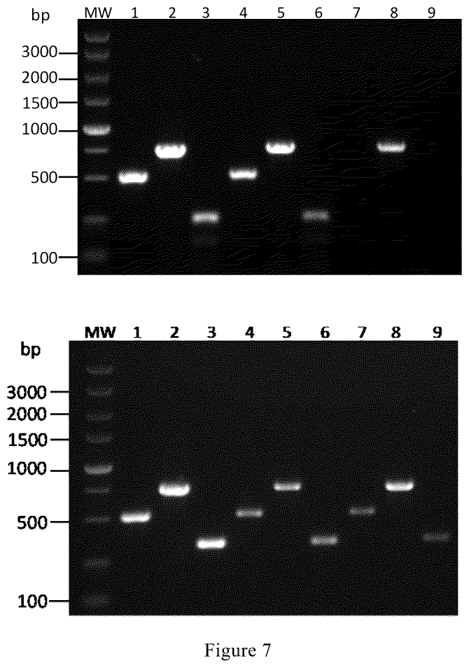

FIG. 6 shows the schematic of primer design for the exemplary PCI-AAV vector impurity DNA PCR analysis. Four pairs of primers were designed. The first pair, F1 and R1, was used to detect impurity DNA upstream of the left ΔDtrsITR of the pciAAV packaging plasmid. The second pair, F2 and R2, was used to detect impurity DNA downstream of the right ΔDtrsITR of the pciAAV packaging plasmid. The third pair, F3 and R3, was used to detect impurity DNA upstream of the left ITR of the rAAV packaging plasmid. The fourth pair, F4 and R4, was used to detect impurity DNA downstream of the right ITR of the rAAV packaging plasmid.

FIG. 7 shows the results of PCR analysis of impurity DNA in the pciAAV vector. It can be observed that the pciAAV vector packaging does not package plasmid backbone impurity DNA molecules into the AAV capsid, resulting in no PCR product. The pciAAV contains only the target DNA band (A, 8) and does not contain detected impurity DNA (A, 7, 9). In contrast, traditional rAAV vector packaging incorporates plasmid backbone impurity DNA molecules on both the left and right sides into the rAAV capsid, forming rAAV vector impurity DNA molecules. PCR amplification with two pairs of primers produced products, showing both the target DNA band (B, 8) and detected impurity DNA (B, 7, 9).

Analysis of Gene Expression in HEK293 Cells Infected by pciAAV Vectors

HEK293 cells were plated in 24-well plates at a cell density of 1.5×10⁵. The following day, the cells were transfected with rAAV6-CMV-EGFP-PolyA (rAAV6-EGFP) and pciAAV6-CMV-EGFP-PolyA (pciAAV6-EGFP) at MOIs of 1×10³ and 1×10⁴, respectively. On the third day post-transfection, green fluorescence in the cells was observed using a fluorescence microscope, and photographs were taken. The percentage of green fluorescent cells and the intensity of green fluorescence were analyzed using flow cytometry.

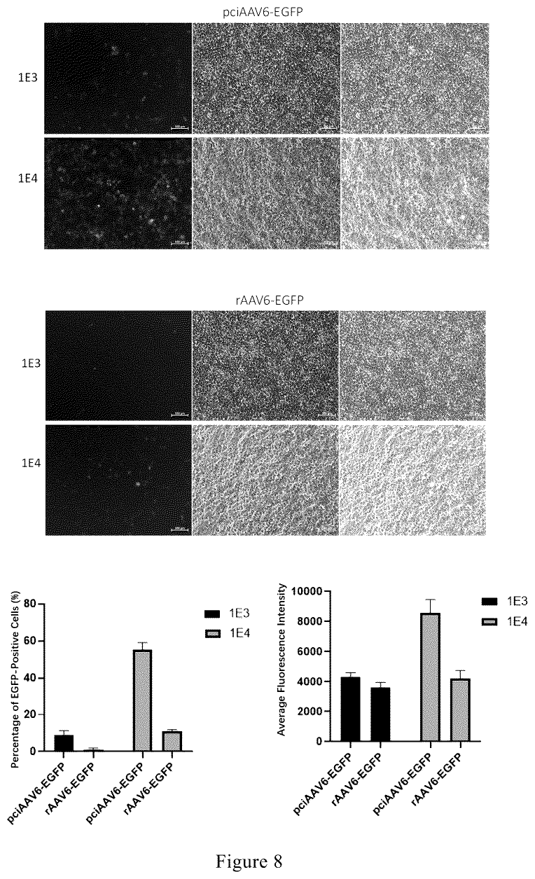

FIG. 8 shows the results of gene expression analysis in HEK293 cells infected with pciAAV vectors. Due to the absence of interference from impurity DNA, the transfection efficiency of pciAAV vectors in cells was higher than that of traditional rAAV vectors, and the green fluorescence intensity was stronger.

What is claimed is:

1. An unpackaged pciAAV genome, comprising:
(a) a modified ITR that lacks a D element and a trs sequence;
(b) a gene of interest or a protection sequence;
(c) a complete ITR;
(d) a gene of interest or a protection sequence; and
(e) a modified ITR that lacks a D element and a trs sequence;
wherein at least one of segments (b) and (d) comprises a gene of interest.

2. The unpackaged pciAAV genome according to claim 1, comprising segments (a) to (e) in 5′ to 3′ direction.

3. The unpackaged pciAAV genome according to claim 1, wherein the unpackaged pciAAV genome comprises a positive single-stranded DNA sequence of the gene of interest and/or a negative single-stranded DNA sequence of the gene of interest between segments (a) and (c) and/or between segments (c) and (e).

4. A pciAAV transgenic plasmid, comprising the unpackaged pciAAV genome according to claim 1.

5. One or more pciAAV vectors, each comprising:
a capsid protein; and
a pciAAV genome packaged by the capsid protein;
wherein the pciAAV genome packaged by the capsid protein is derived from the unpackaged pciAAV genome according to claim 1.

6. One or more pciAAV vectors, each comprising:
a capsid protein; and
a pciAAV genome packaged by the capsid protein; and
wherein the pciAAV genome packaged by the capsid protein is derived from segments (a)-(c) or segments (c)-(e) of the unpackaged pciAAV genome according to claim 1.

7. The one or more pciAAV vectors according to claim 5, further comprising at least a first pciAAV vector and a second pciAAV vector;
wherein the first pciAAV vector and the second pciAAV vector each comprise a complete ITR at one end and a modified ITR at the other end.

8. A method for packaging a pciAAV vector, comprising,
transforming DH10Bac competent E. coli cells with the pciAAV transgenic plasmid according to claim 4 and with Cap and Rep expression plasmids;
performing at least one round of blue-white colony screening, picking white colonies, amplifying, and extracting recombinant bacmids;
transfecting insect cells with the recombinant bacmids to produce recombinant baculovirus; and
extracting the recombinant baculovirus and infecting insect cells with the recombinant baculovirus to obtain the pciAAV vector.

9. An isolated host cell comprising the one or more pciAAV vectors according to claim 5.

10. (canceled)

11. A method for delivering a gene of interest (GOI) to cells, comprising:
contacting the cells with the one of more pciAAV vectors, each comprising:
a capsid protein; and
a pciAAV genome packaged by the capsid protein;
wherein the pciAAV genome packaged by the capsid protein is derived from the unpackaged pciAAV genome according to claim 1.

1. An unpackaged pciAAV genome, which comprises the following, in sequence:
(a) a modified ITR that lacks a D element and a trs sequence;
(b) a gene of interest or a protection sequence;
(c) a complete ITR;
(d) a gene of interest or a protection sequence; and
(e) a modified ITR that lacks a D element and a trs sequence;
wherein at least one of segments (b) and (d) comprises a gene of interest.

2. The unpackaged pciAAV genome according to claim 1, which comprises segments (a) to (e) in 5′ to 3′ direction.

3. The unpackaged pciAAV genome according to claim 1, wherein the unpackaged pciAAV genome comprises a positive single-stranded DNA sequence of the gene of interest and/or a negative single-stranded DNA sequence of the gene of interest, for example, between segments (a) and (c) and/or between segments (c) and (e).

4. A pciAAV transgenic plasmid, comprising the unpackaged pciAAV genome according to claim 1.

5. A pciAAV vector comprising:
a capsid protein; and
a pciAAV genome packaged by the capsid protein;
wherein the pciAAV genome packaged by the capsid protein is derived from the unpackaged pciAAV genome according to claim 1.

6. The pciAAV vector according to claim 5, wherein the pciAAV genome packaged by the capsid protein is derived from segments (a)-(c) or segments (c)-(e) of the unpackaged pciAAV genome according to claim 1.

7. The pciAAV vector according to claim 5, being a group of pciAAV vectors comprising at least a first pciAAV vector and a second pciAAV vector;
wherein the first pciAAV vector and the second pciAAV vector each comprise a complete ITR at one end and a modified ITR at the other end.

8. A method for packaging a pciAAV vector, comprising,
transforming DH10Bac competent E. coli cells with the pciAAV transgenic plasmid according to claim 4 and with Cap and Rep expression plasmids, respectively;
performing at least one round of blue-white colony screening, picking white colonies, amplifying, and extracting recombinant bacmids;
transfecting insect cells with the recombinant bacmids to produce recombinant baculovirus; and
extracting the recombinant baculovirus and infecting insect cells with the recombinant baculovirus to obtain the pciAAV vector.

9. An isolated host cell comprising the one or more pciAAV vectors according to claim 5.

10. Use of one or more pciAAV vectors according to claim 5 in gene therapy, gene editing, or gene regulation.


SEQUENCE LISTING (22 sequences)

SEQ ID NO: 1 - DNA, 34 bp, unidentified
  source 1..34: mol_type = other DNA
cacgtgcggaccgagtgcatggtccggatgccca

SEQ ID NO: 2 - DNA, 34 bp, unidentified
  source 1..34: mol_type = other DNA
gccgctcggtccgagggtacaaggcagggcctgc

SEQ ID NO: 3 - DNA, 39 bp, unidentified
  source 1..39: mol_type = other DNA
agcaccagtcgcggccgctttagatccgaaccagataag

SEQ ID NO: 4 - DNA, 42 bp, unidentified
  source 1..42: mol_type = other DNA
ggccaacctaggagggctgctagcaccagtcgcggccgcttt

SEQ ID NO: 5 - DNA, 33 bp, unidentified
  source 1..33: mol_type = other DNA
gggaaagccggcgaacgtggcgagaaaggaagg

SEQ ID NO: 6 - DNA, 33 bp, unidentified
  source 1..33: mol_type = other DNA
tagtcgacgcgtagtgcatggtccggatgccca

SEQ ID NO: 7 - DNA, 33 bp, unidentified
  source 1..33: mol_type = other DNA
aactagacgcgtagggtacaaggcagggcctgc

SEQ ID NO: 8 - DNA, 33 bp, unidentified
  source 1..33: mol_type = other DNA
tttgtagctagcctagttattaatagtaatcaa

SEQ ID NO: 9 - DNA, 36 bp, unidentified
  source 1..36: mol_type = other DNA
tctaaagcggccgctacaaaatcagaaggacaggga

SEQ ID NO: 10 - DNA, 33 bp, unidentified
  source 1..33: mol_type = other DNA
agggctgctagcagtgcatggtccggatgccca

SEQ ID NO: 11 - DNA, 33 bp, unidentified
  source 1..33: mol_type = other DNA
aactaggctagcagggtacaaggcagggcctgc

SEQ ID NO: 12 - DNA, 35 bp, unidentified
  source 1..35: mol_type = other DNA
tttgtagcggccgcattcttctagagctccatggt

SEQ ID NO: 13 - DNA, 35 bp, unidentified
  source 1..35: mol_type = other DNA
tctaaagcggccgcccggaatattaatagccgcgg

SEQ ID NO: 14 - DNA, 21 bp, unidentified
  source 1..21: mol_type = other DNA
tccgcgttacataacttacgg

SEQ ID NO: 15 - DNA, 20 bp, unidentified
  source 1..20: mol_type = other DNA
gggcgtacttggcatatgat

SEQ ID NO: 16 - DNA, 21 bp, unidentified
  source 1..21: mol_type = other DNA
ttaggtggcggtacttgggtc

SEQ ID NO: 17 - DNA, 21 bp, unidentified
  source 1..21: mol_type = other DNA
cgcagcagggcagtcgcccta

SEQ ID NO: 18 - DNA, 21 bp, unidentified
  source 1..21: mol_type = other DNA
tgattttgtagcggccgcatt

SEQ ID NO: 19 - DNA, 21 bp, unidentified
  source 1..21: mol_type = other DNA
agcgtcgtaagctaatacgaa

SEQ ID NO: 20 - DNA, 21 bp, unidentified
  source 1..21: mol_type = other DNA
atggtgagcaagggcgaggag

SEQ ID NO: 21 - DNA, 21 bp, unidentified
  source 1..21: mol_type = other DNA
ttacttgtacagctcgtccat

SEQ ID NO: 22 - DNA, 20 bp, unidentified
  source 1..20: mol_type = other DNA
gatcataatcagccatacca